(19) United States
(12) Patent Application Publication
(10) Pub. No.: US 2024/0374415 A1
(43) Pub. Date: Nov. 14, 2024

(54) CONFORMAL CONTRACEPTION DEVICES AND METHODS

(71) Applicant: Venova Technologies, Inc., Cambridge, MA (US)

(72) Inventors: Emily Aiko Bao Yee Man, Cambridge, MA (US); Valeria Martinuzzi, Miami Beach, FL (US); Eric K. Lee, Acton, MA (US)

(73) Assignee: Venova Technologies, Inc., Cambridge, MA (US)

(21) Appl. No.: 18/577,751

(22) PCT Filed: Jul. 15, 2022
(86) PCT No.: PCT/US2022/037267

§ 371 Date: Jan. 9, 2024

Related U.S. Application Data
(60) Provisional application No. 63/296,192, filed on Jan. 4, 2022.
(60) Provisional application No. 63/222,932, filed on Jul. 16, 2021.

(51) Int. Cl. A61F 6/14

(52) U.S. Cl. CPC A61F 6/14

(57) ABSTRACT

The present disclosure relates generally to conformal contraceptive devices and, more particularly, to intracervical contraceptive devices. Various embodiments disclosed herein relate to an intracervical conformal contraceptive device that can be configured to be removably secured in the endocervix of the cervical canal. The device, in certain embodiments, comprises a generally tubular or cylindrical structure defining a conduit extending from a proximal (uterine) end of the device to an opposite distal (vaginal) end of the device. The structure may, in some cases, be compliant and/or configured to conform to and/or securely engage the walls of the endocervix to inhibit inadvertent movement of the device out of the cervical canal. In some cases, the device may be equipped with a self-closing one-way valve in the conduit, which allows flow of menstrual fluid from the uterine cavity through the conduit to the vagina, while inhibiting flow of seminal fluid resulting from coitus from the vagina through the conduit to the uterine cavity. Other embodiments are generally directed to systems and methods for making or using such devices, kits including such devices, or the like.

### RELATED APPLICATIONS

This application claims the benefit of U.S. Provisional Patent Application Ser. No. 63/222,932, filed Jul. 16, 2021, entitled “Conformal Contraception Devices and Methods,” and of U.S. Provisional Patent Application Ser. No. 63/296,192, filed Jan. 4, 2022, entitled “Conformal Contraception Devices and Methods.”

### FIELD

The present disclosure relates generally to conformal contraceptive devices and, more particularly, to intracervical contraceptive devices.

### BACKGROUND

Unintended pregnancy remains a global women's health issue. Female contraception is fundamental to reducing the rate of unintended pregnancies, and essential for effective family planning and improving the social and economic well-being of women around the world. A variety of contraceptive methods are currently available including single-use barrier methods (cervical cap, diaphragm, and condoms), short-acting hormonal methods (oral pills, patch, ring, and injection), long-acting reversible contraceptives (LARCs; intrauterine devices (IUDs) and the birth control implant), and female sterilization. Each method varies in its effectiveness, convenience, cost, user adherence, and side-effect profile. However, highly effective birth control, greater than 99% effective, is restricted to permanent sterilization, hormonal LARC methods, and the non-hormonal copper IUD, all of which are associated with potentially adverse health implications.

Single-use barrier contraceptive methods rely on user compliance, disciplined administration to function properly, and are overall the least effective compared to other contraceptive measures. For example, the cervical cap is a deep silicone cup inserted into the vagina and adheres to the cervix by suction or by constriction. Insertion and removal of this device can be difficult for some users and may require a health care provider, e.g., a medical professional or practitioner, to ensure proper placement. Cervical caps are not necessarily secure during intercourse, reducing the contraceptive efficacy. Similarly, the diaphragm is a dome shaped cup that expands at the top of the vagina to cover the cervix. Both the cervical cap and the diaphragm require the use of a spermicidal agent to be effective. However, frequent use of spermicidal agents can irritate the lining of the vagina, potentially increasing the risk of contracting HIV and other sexually transmitted diseases. Condoms are another single-use barrier device. Condoms require proper preparation and application with each instance of intercourse and may be subject to a moderately high failure rate if used improperly or inconsistently.

Short-acting hormonal methods vary in terms of effectiveness and are dependent on synthetic hormonal agents, frequent administration, and consistent user compliance. Oral contraceptives (the “pill”) are popular but require the user to adhere to a daily administration schedule. The transdermal birth control patch delivers hormonal agents through the skin and needs to be replaced every week on the same day. The vaginal ring is placed around the cervical neck and delivers hormones over a three-week period. The ring can be inadvertently dislodged by a sexual partner during intercourse or can migrate downwards and exit the body unintentionally due to its accessible location at the top of the vagina. The birth control shot is a subcutaneous injection of hormonal agents administered every three months. This method allows for a longer dosing interval but requires a trained healthcare professional to administer the injection correctly. All of the short-acting methods described above present similar risks of unwanted side effects due to the use of synthetic hormones.

Long-acting reversible contraceptives (LARCs), which include intrauterine devices (IUDs) and hormonal implants, carry risks based on their design, method of deployment, or the use of pharmacological agents. IUDs function by interfering with the implantation of the fertilized egg on the endometrium. The basic designs comprise a T-shaped structure with either a copper component or with a reservoir of hormonal agent, typically progestin levonorgestrel. In the case of the non-hormonal IUD, copper (or another metallic variant) is used as a spermicide agent because it is toxic to sperm and inhibits sperm from fertilizing an egg. However, copper can induce an inflammatory response, cause host intolerance, and alter the natural menstrual cycle.

Hormonal IUDs and birth control implants, similarly to short-acting hormonal methods, use a combination of hormonal agents to regulate the physiology of the uterine lining in order to prevent pregnancy. IUDs and implants must be inserted by a healthcare professional. After deployment of the device into the uterus, strings from the device suspend down into the vagina to allow for future removal when the service life expires or when the user plans to conceive. Some users experience discomfort during intercourse due to the protrusion of the retrieval strings into the vagina. IUDs in general are subject to dislocation, cither expulsion into the vagina, or into the uterus where the device could puncture the uterus. To ensure correct IUD placement, users may be advised to visit their health professionally annually and undergo ultrasounds, an imaging procedure that is expensive and requires advanced planning. In the case of the birth control implant, a small rod is implanted subcutaneously on the upper arm and releases hormones at a controlled rate. The implant is also subject to dislocation and migration to other parts of the body. The implant can be seen and felt beneath the skin of the arm, which may concern some users. For both the hormonal IUD and the implant, the user may experience side effects such as weight gain, worsened cramps, nausea, dermatological disorders, and irregular bleeding during the first few months of implantation.

Recently, concern regarding extended use of any type of hormonal contraceptive has increased based on research associating long-term hormonal use with increased incidence of tumor growth. Although cysts form and change naturally on the breasts and ovaries during the course of a menstrual cycle, extended use of hormonal contraceptives (primarily longer than 10 years) has been associated with elevated risk of breast cancer. There is also concern with respect to how hormonal changes may affect mood, and the desire of many women to eliminate synthetic hormones and preserve a natural menstrual cycle.

Female sterilization is a permanent method of female contraception. Surgical sterilization procedures include tubal ligation (also known as tube tying) and hysterectomy. These options are expensive, require an extended recovery period, and can pose potential health complications including damage to the bladder, rectum, or other pelvic structures. In addition, the irreversible effect is undesirable for women of child-bearing age whose birth control options should accommodate changing needs and circumstances.

Fallopian tube occlusion is a nonsurgical method of female sterilization using a small coil or other insert placed in the fallopian tubes. Over a period of time, scar tissue develops around the insert blocking the tubes. Several devices were introduced to the market but have since been recalled by the FDA because of numerous complications regarding device migration, unreliable permanent contraception, and adverse effects such as increased external and internal bleeding.

Other device concepts have been proposed in the prior art to address various issues of commercial female contraceptives. Others have components that span the cervical canal or protrude into the vagina or uterus, which adversely affect usability, risk of injury, or discomfort during sexual intercourse. In some device concepts, one-way valves are introduced to the design of cervical caps to be located in the vagina similar to commercial cervical caps, such as U.S. Pat. Nos. 4,848,363. 3,952,737 and 2,836,177 similarly describe a device that is secured around the neck of the cervix and slightly protrudes into the cervix while enclosing a one-way valve mechanism. These devices are intended to permit outflow material and prevent inflow material into the cervix. However, these devices are subject to the similar deficiencies as marketed cervical caps, including unintended dislodging, low efficacy rates if not placed properly and without spermicides, and risk of infection and toxic shock syndrome if not cleaned or replaced regularly.

Some devices operate by allowing outward flow of menstrual fluids while blocking inward flow of sperms. In some device concepts, the device is positioned in the cervical canal and contains a valve that allows flow from the uterine cavity into the vagina, but not in the reverse direction. U.S. Pat. No. 4,146,024, for example, describes a design that involves a tubular member, inside of which are a valve stem, a spherical valve member, or a dished valve member. The complexity of this device concept yields difficult implementation and monitoring due to its reliance on multiple small moving parts to function fully. Mechanical parts could be clogged by semi-solids often present, leading to unreliable operation of the valve mechanism if it does not readily return to its original position. This results in compromised menstrual drainage and contraception functions. Similarly, in U.S. Pat. No. 4,004,582, a complex one-way valve system is described incorporating a pharmacological agent to further disrupt fertilization as a secondary measure of contraception. However, this device concept includes attached members that extend into the uterine cavity to prevent inadvertent expulsion from the body. Significant disruption to the uterine tissues and passage through the junction between the uterus and cervix can induce discomfort for the uterus and compromise the natural tissue strength.

In another intracervical device concept, U.S. Pat. No. 4,932,422 describes a cylindrical body to be retained within the cervix having a valve mechanism adapted to allow passage of menstrual fluid yet blocking seminal fluid. The device relies on hooks protruding out of the cervix and engaging with the inner bottom wall of the uterus to retain the mechanism in place. The device involves multiple moving mechanisms including a hinging flap and a spring, or multiple flaps and multiple springs. These mechanisms are necessarily small and delicate, which detract from reliability. This device concept also engages with the junction between the cervix and uterus to potentially compromise the surrounding tissues and induce pain and discomfort.

The contraceptive concepts mentioned above all exhibit limitations and complexities. There is a need for an alternative to permanent sterilization that is safe and non-surgical. There is a need for a contraception solution that can be deployed indefinitely, but with the option to be reversed to accommodate changing fertility decisions. There is a need for a long-acting contraceptive that does not have a predetermined service life. There is a need for a nonhormonal device that does not disrupt the natural flow of menstrual cycles nor interrupt uterine and surrounding tissues. There is a need for a nonhormonal device that has minimal adverse reactions and a reduced side effect profile. In addition to addressing these unmet clinical needs, an improved contraceptive solution would deliver the convenience, reliability, and low-maintenance user experience expected by a diverse user population.

### BRIEF SUMMARY

The present disclosure relates generally to conformal contraceptive devices and, more particularly, to intracervical contraceptive devices. The subject matter of the present disclosure involves, in some cases, interrelated products, alternative solutions to a particular problem, and/or a plurality of different uses of one or more systems and/or articles.

An intracervical conformal contraceptive device, in accordance with one or more embodiments, can be configured to be removably situated in the endocervix of the cervical canal. The device, in some embodiments, comprises a generally tubular structure defining a conduit therein extending from a uterus end of the device to an opposite vaginal end of the device. The generally tubular structure may, in some cases, be compliant and/or configured to conform to and securely engage the walls of the cervical canal to inhibit inadvertent movement of the device out of the cervical canal. In some cases, the device may also include a conduit encasing at least one unidirectional flow control component (e.g., a valve) for allowing flow of menstrual fluid from the uterine cavity through the conduit to the vagina, while inhibiting flow of seminal fluid, if present, from the vagina through the conduit to the uterine cavity.

One aspect is generally directed to an intracervical conformal contraceptive device configured to be removably plugged in the cervical canal. In one embodiment, the device comprises a generally tubular structure defining a conduit therein extending from a uterus end of the device to an opposite vaginal end of the device, said generally tubular structure being compliant and configured to conform to and securely engage the walls of the cervical canal to inhibit inadvertent movement of the device out of the cervical canal, and a one-way valve positioned in the conduit for enabling flow of menstrual fluid from the uterine cavity through the conduit to the vagina, while inhibiting flow of seminal fluid from the vagina through the conduit to the uterine cavity.

Another aspect is generally directed to an intracervical conformal contraceptive device. In one set of embodiments, the device comprises a housing defining a conduit passing from a first end to a second end of the housing. In some cases, the housing comprises an outer surface and features that may vary in rigidity, softness, compliance, etc. In certain embodiments, the housing comprises a one-way valve within the conduit.

In another set of embodiments, the intracervical conformal contraceptive device, comprising a housing defining a conduit passing from a first end to a second end of the housing. In some cases, the housing comprising an outer surface formed from a material having a Young's modulus of no more than 5 GPa. In certain embodiments, the housing comprises a one-way valve within the conduit.

The intracervical conformal contraceptive device, in yet another set of embodiments, comprises a housing having a cervical shape, defining a conduit passing from a first proximal end to a second distal end of the housing. In some cases, the housing comprises a one-way valve within the conduit. In certain embodiments, the one-way valve allowing fluid to pass from the first proximal end to the second distal end.

In another set of embodiments, the intracervical conformal contraceptive device comprises a housing that exhibits a cylindrical shape and that fits within the cervix, defining a conduit extending from a proximal (uterine) end of the device to an opposite distal (vaginal) end of the device. In some embodiments, the device comprises a flow control component (e.g., a one-way valve) within the conduit, allowing fluid to pass from the uterus to the vagina. Yet another aspect is generally directed to a method comprising intravaginal insertion and deployment into the cervix.

In addition, certain aspects are generally directed to a method. In one set of embodiments, the method comprises inserting, into a vagina of a subject, a device comprising a housing defining a conduit passing from a first end to a second end of the housing. In some cases, the housing comprises a one-way valve within the conduit, such that the one-way valve is positioned to allow fluid to exit the vagina.

In another aspect, the present disclosure encompasses methods of making one or more of the embodiments described herein, for example, a contraceptive device. In still another aspect, the present disclosure encompasses methods of using one or more of the embodiments described herein, for example, a contraceptive device.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 illustrates placement of an exemplary intracervical contraceptive device in accordance with one or more embodiments in the cervical canal.

FIGS. 2A-2C and 3-5 illustrate one basic construction of an exemplary intracervical contraceptive device in accordance with one or more embodiments.

FIGS. 6, 7, 8A-8D, and 9 illustrate various attachment mechanisms in accordance with one or more embodiments.

FIGS. 10-15 illustrate exemplary valves that may be used in intracervical contraceptive devices in accordance with one or more embodiments.

FIGS. 16A-16F illustrates exemplary reinforcement features in accordance with one or more embodiments.

FIGS. 17A-17H, 18A-18F, 19A-19D, and 20A-20D illustrate methods of use in accordance with one or more embodiments.

FIG. 21 illustrates a method of insertion, in accordance with one or more embodiments.

### DETAILED DESCRIPTION

The present disclosure generally relates to a contraceptive device that is inserted intravaginally and positioned in the cervix of a female subject. The device allows the flow of menstrual fluid from the uterine cavity (within the uterus) into and out of the vagina, while inhibiting the flow of seminal fluid from the vagina into the uterine cavity. Accordingly, the device is able to prevent pregnancy by preventing the entry of seminal fluid into the uterus, while still allowing menstrual flow to occur.

The present disclosure relates generally to conformal contraceptive devices and, more particularly, to intracervical contraceptive devices. Various embodiments disclosed herein relate to an intracervical conformal contraceptive device that can be configured to be removably secured in the endocervix of the cervical canal. The device, in certain embodiments, comprises a generally tubular, spindle, or cylindrical structure defining a conduit extending from a proximal (uterine) end of the device to an opposite distal (vaginal) end of the device. The structure may, in some cases, be compliant and/or configured to conform to and/or securely engage the walls of the endocervix to inhibit inadvertent movement of the device out of the cervical canal. The cervix is typically around 2-4 cm in length, and the device may thus have a similar length in some embodiments. On the interior (proximal) end of the subject, the cervix communicates with the uterus (or internal os) via an internal orifice, while on the exterior (distal) end, it communicates with the vagina via an external orifice. The internal orifice of the uterus is an interior narrowing of the uterine cavity. It corresponds to a slight constriction known as the isthmus that can be seen on the surface of the uterus about midway between the apex and base. The external orifice of the uterus is a small, depressed, somewhat circular opening on the rounded extremity of the cervix. opening to the vagina. The external orifice is bounded by two lips, an anterior and a posterior. The anterior is shorter and thicker, though it projects lower than the posterior because of the slope of the cervix. Normally, both lips are in contact with the posterior vaginal wall. Prior to pregnancy, the external orifice has a rounded shape when viewed through the vaginal canal.

The device may have, in some embodiments, a conduit passing from one end to the other, e.g., defined within a housing, such as a cylindrical housing. In some cases, the conduit is generally tubular, spindle, or cylindrical. The conduit may also contain a flared open in some embodiments, e.g., on the interior end and/or on the exterior end of the conduit. This conduit may be designed to allow the flow of fluid. However, in some cases, the conduit may allow fluid to flow in a direction from the interior end towards the exterior end, relative to the opposite direction. This may be useful, for example, to allow menstrual fluids to exit from the uterine cavity, while substantially preventing or inhibiting sperm or seminal fluid to enter into the uterine cavity, thereby preventing or inhibiting pregnancy. In some cases, the device may be equipped with a flow control component in the conduit, which allows flow of menstrual fluid from the uterine cavity through the conduit to the vagina, while inhibiting flow of seminal fluid resulting from coitus from the vagina through the conduit to the uterine cavity. Other embodiments are generally directed to systems and methods for making or using such devices, kits including such devices, or the like.

In one set of embodiments, fluid may flow preferentially in one direction through the conduit. In some cases, this may be accomplished using a one-way mechanism, e.g., a valve, a normally closed flap that opens for fluid egress, etc., that can be positioned partially or completely within the conduit. In some embodiments, however, a portion of the one-way valve may not be present within the conduit. The one-way valve may be configured in certain cases to be normally closed, but to open under the hydraulic pressure of menstrual fluid or related discharge from the uterus. In some cases, the one-way valve may be self-closing. The valve may also be designed to revert to a closed position once the menstrual fluid is substantially cleared from the uterine cavity in some cases.

Various types of one-way valves can be used in various embodiments including, but not limited to, valves comprising a plurality of flaps such as those in bicuspid valves (commonly referred to as duckbill valves) and tricuspid valves. Such valves may have 2 (i.e., bicuspid), 3 (i.e., tricuspid), 4 (i.e., “cross-slit”), 5, 6 or any other suitable numbers of flaps. In some cases, the flaps may be substantially flexible, e.g., such that when pressure is applied in one direction, the flaps diverge to create an opening for flow to occur, while when pressure is applied in the opposite direction, the flaps may be pressed together to form a liquid-tight seal. Other non-limiting examples include drain valves, check valves that open and close under spring tension, ball valves that operate within a restricting cage, tricuspid valves that mimic anatomical opening and closing of heart valves, and other valves comprising flaps or as seen in mechanical heart valve implants. These valves may allow the forward flow of fluid as the flaps open under pressure, but close when the pressure subsides, or when a pressure (e.g., a fluid pressure) in the reverse direction is applied. In some cases, the valves can form a water-tight or liquid-tight seal when pressure in the reverse direction is applied. Menstrual fluid and related discharge can thus exit the uterine cavity as a result of the natural shedding of the uterine lining and consequential intrauterine pressure buildup inside the uterus. Intrauterine pressures during the menstrual cycle generally range from 10 to 50 mmHg. The one-way valves may be designed in certain embodiments to open at hydrostatic pressures under the intrauterine pressure at any stage of the menstruation cycle, and/or to close against reverse flow at reverse pressures higher than those encountered during intercourse, thereby preventing passage of semen into the uterus.

In addition, in certain embodiments, more than one such one-way valve may be present, and if more than one valve is present, the valves may be the same or embody different properties. In addition, in one or more embodiments, the device includes a plurality of one-way valves in tandem within the device conduit. Having multiple valves may increase contraceptive reliability, for example, by reducing the likelihood of seminal fluid intrusion through the values.

In some cases, devices such as those described herein may contain one or more features that allow them to be secured within the subject. For instance, in one embodiment, a device may have a shape that substantially conforms to the cervix. In some cases, a device may be designed to seal the cervical cavity of a subject such that no liquid can bypass outside or around the device while inside the cervical canal. For example, a device may have a nominal diameter at least equal to or slightly larger than that of the cervical canal, and/or the length of the device may be designed to fit substantially inside the cervical canal. Variations in length in different embodiments may be used for proper fit of different anatomical characteristics. In one or more embodiments, the device may have dimensions to fit much of the intended user population. Based on an average cervical canal length and a nominal diameter that varies by a few millimeters, the device may have a length of three centimeters (3 cm) or less, and five millimeters (5 mm) or less in overall diameter. However, a range of dimensions is also possible in different embodiments, including smaller or larger than these dimensions. For instance, in one set of embodiments, the device may have a length of no more than 6 cm, no more than 5 cm, no more than 4 cm, no more than 3 cm, or no more than 2 cm. In some cases, the device may have an outer diameter of no more than 15 mm, no more than 14 mm, no more than 13 mm, no more than 12 mm, no more than 11 mm, no more than 10 mm, no more than 9 mm, no more than 8 mm, no more than 7 mm, no more than 6 mm, or no more than 5 mm.

In some cases, the natural compliance of the cervical canal may exert a force on a device to contain the device within the subject. For example, in one or more embodiments, a device may be pre-compressed before it is inserted in the cervical canal and allowed to expand once positioned properly to engage with the cervical wall so that it is securely retained in place. Self-expansion of the device may be achieved by sizing the device, for example, in its expanded form according to the size of the cervical canal such that sufficient radial contact force is exerted against the cervical wall to reduce the likelihood of dislocation. As another example, the device may include a self-expanding stent on its outer surface, which may be able to adapt to the shape and size of a given user's anatomy once implanted in the cervical canal. In some cases, e.g., when the device needs to be removed or replaced, the stent may be contracted using a filament mechanism to compress the device, thus releasing it from the wall of the cervical canal for retrieval.

In another set of cases, the device may be secured within the cervical canal by using or enhancing adhesion between the outer wall of the device and the tissue lining of the cervix. As yet another example, the device may include one or more adhesives (or glues) that allow the device to be secured. Non-limiting examples of adhesives include chitosan, hydrogels, fibronectin, fibrin, thrombin, shellac, or the like. Many such bio-adhesives are known and readily available. Adhesion may be achieved in some embodiments through non-toxic and/or biocompatible glues that are applied to the outer surface of the conduit to attach to the cervix. In some cases, the adhesive may be responsive to pH. In certain embodiments, the material of the device can act as an adhesive in the presence of moisture or pH specific to the cervical tissue. The pH-responsive material may be adaptable in some embodiments to the variance of pH during the menstrual cycle.

Features can be incorporated on the outside of the device in certain embodiments to help secure its position inside the cervical canal. These may in some cases contact and engage the cervical wall, which may help to anchor such devices in place. In some embodiments, the device may be secured once it is placed in the cervix. In some cases, the device may have features that prevent sperm passage around the device. In some embodiments, the device may be retrievable after insertion. In some cases, the device may have external features, e.g., retention mechanisms such as ridges, bumps or other sections having an enlarged diameter, textured surfaces, vanes (e.g., circumferentially positioned), etc. on the outside surface of the device.

In various embodiments, these external features can be configured as unidirectional, bidirectional, or non-directional. In one or more embodiments, these features may wrap partially or fully around the circumference of the device. In one or more embodiments. unidirectional ridges are located (but are not limited to) the proximal or distal ends of the device. In some embodiments, the external features may have rounded edges and may vary in thickness, location, length, etc. In some embodiments, a singular rib or a plurality of ribs may spiral partially or fully around the circumference of the device. Circumferential, continuous (e.g., non-spiral) flexible features deployed singly, or in tandem in some cases, may serve as a barrier against liquid passage, e.g., in a manner analogous to squeegees or windshield wipers on automobiles. The increased contact between the device features and the cervical wall may help establish a seal that minimizes or reduces semen/sperm bypass. Furthermore, the dimension, number, spacing among the features, aspect ratios of the features, etc., vary in some embodiments between different devices, for example, to balance fit, function, and comfort in different human subjects. For instance, in one embodiment, different devices may be prepared having different size features, e.g., to fit a range of cervical dimensions of different subjects. In some embodiments, a device housing may include non-isotropic diameters along their lengths. In some embodiments, a device may have various longitudinal and radial pressure profiles, or other features, e.g., to allow precise placement, positional stability once the device is in place, prevention against sperm leakage, etc.

In some embodiments, one or more external features can be deflected as needed, for example, to conform to the cervical canal. For instance, cervical canals are not necessarily perfectly cylindrical and may have folds or other surface irregularities. In some embodiments, the device may have one or more ridges, bumps, vanes, etc. These may be useful, for example, as redundant contact points that exert significant local pressure at contact points with the cervical wall.

Owing to their flexibility, individual features may deform independently in some embodiments in response to local variations of topology or temporal changes in dimension resulting from contractions or similar events. They may also be able to return to their original positions in some cases.

In some embodiments, the external features and the housing can be made from the same material. In some other embodiments, the materials of the external features and housing can be made from different materials. The sheath and the body of the device can also be made from the same or different materials. In one set of embodiments, the features are present as an outer sheath on a device, and can be also be made from the same or different materials. In some embodiments, a single cylindrical device body may comprise multiple sheaths, for example, with various external feature dimensions and various materials.

In another set of embodiments, the device includes a textured outer surface that increases the friction between the device and the cervical canal. A variety of surface textures may be used to reduce slippage or sliding of the device. The increase in friction is a function of the surface roughness measured by the rms (root mean square) height of the surface bumps or texture. Suitable surfaces include matte (resembling ground glass) to fine knurling. In some cases, the rms height of the textured surface may range between 1 mm and 50 mm, e.g., corresponding to the texture of ground glass and fine knurling, respectively. In some cases, the rms height may be at least 1 mm, at least 2 mm, at least 3 mm, at least 5 mm, at least 10 mm, at least 15 mm, at least 20 mm, at least 25 mm, or at least 30 mm, and/or no more than 50 mm, no more than 40 mm, no more than 30 mm, no more than 25 mm, no more than 20 mm, no more than 15 mm, no more than 10 mm, no more than 5 mm, no more than 3 mm, no more than 2 mm, etc. Combinations of any of these are also possible in certain embodiments. Surface texturing can also be used with other devices as discussed herein (for example, with protrusions mentioned above, etc.).

In one or more embodiments, a device may include a flared or flanged end, e.g., at the vaginal end and/or at a uterine end. The flared opening may extend out of the opening of the cervix into the vagina. This flared opening may be used to serve one or more purposes. These include conforming to the shape of the external os, e.g., to ensure comfort and minimize exposed edges; limiting the depth of insertion, e.g., such that the device is properly located inside the cervical canal but does not move into the uterus; and/or (3) preventing migration of the device into the uterus during sexual intercourse due to pressure build up in the vagina, etc.

The device, or at least a portion of the device, may be formed from suitable materials in various embodiments. For example, the materials may be non-toxic (e.g., cytotoxic) and/or biocompatible, for example, in the cervical environment and/or the surrounding reproductive tissues. The device may be formed from a single material, or from two or more materials, including any of the materials described herein. As a non-limiting example, in one set of embodiments, the device may comprise a valve (e.g., a one-way valve such as described herein) from a first material, and a conduit containing the valve formed from a second material. Non-limiting examples of such materials include hydrophilic polymers or copolymers, grafted polymers, hydrophobic polymer surface-modified to render them hydrophilic, etc. Non-limiting examples of suitable polymers include polyether ether ketone, polyetherimide, polyethersulfone, polysulfone, polyphenylsulfone, polyethylene, ultra-high molecular weight polyethylene, elastomers (e.g., polydimethylsiloxane, polyurethane, etc.), or the like.

In some embodiments, the device may include an outer surface that allows the device to substantially conform to the cervix. For example, the outer surface of the device may include a relatively conformable material. Examples include, but are not limited to, polycarbonate-urethane, silicone, a hydrogel, an elastomer (e.g., polydimethylsiloxane, polyurethane, etc.), or the like. In some cases, the outer surface comprises a material having a Young's modulus of no more than 100 GPa, no more than 50 GPa, no more than 30 GPa, no more than 10 GPa, no more than 5 GPa, no more than 3 GPa, no more than 1 GPa, no more than 0.5 GPa, no more than 0.3 GPa, no more than 0.1 GPa, etc.

In some embodiments, the device may comprise one or more than one material of construction for the purpose of modifying the physical properties of the device, such as its modulus and/or viscoelasticity. In certain embodiments, for example, the device may comprise a material that provides structural strength and another material that provides biocompatible contact surfaces with cervical tissue. In certain embodiments, a wall of the device may comprise a structural composite of multiple materials. For example, in one embodiment, such materials may protect a valve (e.g., a one-way valve) and its flexible flap-like elements from distortion during use. A structural composite may comprise, in some embodiments, one or more polymeric materials of varying modulus. In some embodiments, the structural composite may comprise polymeric and/or inorganic materials including, but not limited to, ceramics and metals.

In addition, in some embodiments, the device may contain, for example, one or more spermicidal and/or hormonal agents and/or metals or metal-variants therein. These may be positioned anywhere within the device, e.g., before and/or after a valve, between two valves, etc. These may act, for example, as contraceptive safeguards. Examples of hormonal agents include, but are not limited to, estrogen (e.g., ethinylestradiol), progestogen (e.g., progestin), etc. Examples of spermicides include, but are not limited to, nonoxynol-9. Octoxynol-9, benzalkonium chloride, sodium chlorate, lactic acid, neem oil, metals (e.g., copper), or the like. Many spermicides and hormonal agents can be obtained commercially. Such agents may be present within the device, for example, as a foam, a film, a jelly, a cream, a gel, a sponge, saturated or embedded in a porous or non-porous material, or the like. For example, in some embodiments, the device may comprise two one-way valves, with a chamber defined between the valves that contains one or more spermicidal and/or hormonal agents therein. In some cases, the chamber may be used as a reservoir for the spermicidal and/or hormonal agents. In some cases, the agent may be contained within a material. In some cases, the material may be shaped to allow flow to pass through; for example, the material may be present in a “lining” within the conduit. The material may be porous or non-porous. The material may, for example, comprise a gel or a sponge.

Devices in accordance with various embodiments may be designed to be located securely in the cervical canal for the intended duration of use wherein the entire device is removable or replaceable as a single unit. For instance, the device may be positioned within a subject for at least a day, at least a week, at least a month, at least 2 months, at least 4 months, at least 6 months, at least 1 year, at least 2 years, at least 3 years, at least 4 years, at least 5 years, etc.

The following embodiments pertain to the deployment of the device into the cervical canal. In one embodiment, the device may be inserted into the cervical canal, without necessarily spanning or intruding the uterine cavity. For example, a device may be deployed with a delivery system similar to the Paragard copper IUD. In some cases, the device may be inserted into the uterine cavity using a guidewire, similar to a stent. For example, in some embodiments, the device may be protected within an insertion tube or an outer sheath of a delivery device and fitted in the cervical canal after deployment, e.g., in a manner similar to the deployment of stents in blood vessels. The outer sheath or insertion tube may enter the vaginal canal to the base of the cervix, and a guidewire may be fed through the outer sheath or insertion tube and inserted through the cervical canal. The guidewire may be extended upward into the uterine cavity where prongs may be exposed. The guidewire may be gently pulled back towards the vagina until the prongs interact with the base of the uterus, and in some cases may act as a measurement tool to indicate the top of the cervix. The outer sheath or insertion tube may be fed up along the guidewire. The device may be fed within the outer sheath or insertion tube along the guidewire, e.g., using a pushing piston. The outer sheath or insertion tube and the guidewire may then be pulled back to expose the device that is held in position with the pushing piston. Once the device has been positioned in the cervix, the pushing piston can be withdrawn with the rest of the delivery system.

In another example, a delivery system may be used to deliver the device with an insertion tube, which can act similarly to the barrel of a syringe. The insertion tube may be designed to fit over the rounded extremity of the cervix. In some cases, the insertion tube may have a rounded end, e.g., to protect the device, and/or to allow for easier insertion, etc. The insertion tube, in some cases, may be formed of a flexible material, where an opening at an end of the insertion tube can be opened or widened, e.g., to allow the device to pass through the opening into the subject. Examples of potentially suitable materials include thermoplastics such as low-density polyethylene, polypropylene, polyethylene terephthalate, polybutylene terephthalate, etc. In some embodiments, the stiffness of the insertion tube may be controlled by material selection and wall thickness. In some embodiments, the materials may be sterilizable. In one embodiment, the insertion tube may be rigid enough to stabilize the device during storage and insertion, but flexible enough to allow a rounded, slitted end to open when the sheath of the insertion tube is withdrawn to expel the device (e.g., as with a tampon).

In some cases, the center of the insertion tube may be aligned with the ostium or external os. A guidewire may be fed through the insertion tube through the cervix and into the uterine cavity. The device may be fed through the insertion tube along the guidewire, and into the cervical canal, e.g., using a plunger. The plunger may push the device past the junction between the cervix and the vagina, and in some cases may conformally expand within the cervical canal. In some embodiments, the insertion tube may be inserted through the vagina, for example, past the external os of the subject or such that it contacts but does not necessarily pass through the internal os of the subject. In some case, the insertion tube may be retracted from the subject, for example, while holding the plunger stationary. For instance, the insertion tube may be withdrawn from the subject in such fashion. Once the device is secured, the delivery system can be removed. In one embodiment of this delivery method, alignment of the delivery system is reliant on the position of the insertion tube at the extremity of the cervix, e.g., exclusively reliant in some cases, such that the device is designed to be properly located when the flared end of the device is seated on the ostium. As with IUD deployment, a speculum may be used to assist with accessing and preparing the cervical area in this procedure. The insertion tube may be designed for initial placement and subsequent re-insertion by a qualified healthcare provider.

In some embodiments, the insertion tube may have a length of at least 5 cm, at least 10 cm, at least 15 cm, or at least 20 cm. In addition, in some embodiments, the insertion tube may have a length of no more than 20 cm, no more than 15 cm, no more than 10 cm, or no more than 5 cm. Combinations of any of these lengths are also possible in certain cases; for instance, the insertion tube may have a length of between 10 cm and 20 cm.

In addition, in some embodiments, the insertion tube may comprise an external flange, a stopper, or other mechanical retention mechanisms. Such mechanisms can be used, in some embodiments, to prevent insertion past the internal os of the subject. In some cases, such mechanisms may also be adjustable.

In another non-limiting example, the device may be deployed without the guidance of a guidewire. As an example, in some cases, the delivery system does not extend through the internal os nor into the uterine cavity. For example, an outer sheath may enter the vaginal canal to the base of the cervix. The delivery system may include a stopper or other indication of measurement to prevent the delivery system from passing beyond the cervical canal through the internal os and into the uterine cavity. The outer sheath may be pulled back to expose the device that is held in position with a pushing piston. Once the device is inserted, the pushing piston may be withdrawn with the rest of the delivery system.

In yet another example, a cylindrical insertion tube may act similarly to the barrel of a syringe without the guidance of a guidewire. The plunger may push the device past the junction between the cervix and the vagina, and conformally expands within the cervical canal. The plunger may then be retracted leaving the device expanded in place. Once the device is secured, the delivery system can be removed.

Examples of devices having some or all these features are now described in more detail below. It should be understood, however, that these features are not mutually exclusive, but are shown in different figures herein for purposes of clarity. It is to be understood that in some embodiments, a device falling within the scope of the claims may comprise two or more features that are shown in different figures herein and discussed within the following text but are not shown within the same figure or described as being used together within the accompanying text, i.e., to reduce potential confusion. However, such embodiments nevertheless should be understood as falling within the scope of the present disclosure. Accordingly, the following references to the figures are for purposes of illustration only and are not intended to be limiting.

For example, FIG. 1 illustrates an exemplary intracervical contraceptive device 10 in accordance with one or more embodiments describing a one-way valve inserted in the cervical canal 12. The device in this non-limiting example permits fluid flow in the direction from the uterus 14 to the vagina 16 but prevents or at least inhibits fluid flow in the opposite direction from the vagina 16 into the uterus 14. The device 10 is located in the cervix in this embodiment, as opposed to the uterine cavity 14 or vagina 16. The location of the device 10 in the cervical canal may be useful to preserve the physiological functions and health of the uterus 14 and the vagina 16. In some cases, the device may enhance the natural purpose of the cervix as the gateway between the two anatomies. In particular, during pregnancy, the cervix naturally closes to seal flow from the vagina 16 to the uterus 14 and prevent infection. The device 10 may mimic and/or reinforce this function to prevent or inhibit fluid flow from the vagina 16 to the uterus 14. For the proper functioning of the device in this particular example, the cylindrical housing 20 of the device may be designed to conform to and seal the cervical cavity 12 such that virtually no liquid can bypass the device 10 and flow outside the cylindrical structure 20 between the uterus 14 and the vagina 16. However, it should be understood that other shapes or configurations are also possible in other embodiments

In this particular non-limiting example, the nominal diameter of the device 10 may be at least equal to or slightly larger than that of the cervical canal 12. Given normal anatomical variability, the cylindrical structure 20 may be constructed in some cases with a material that is conformable to accommodate differences in diameter and length of a given cervix 12 among different users. Devices having a range of diameters may be provided in various embodiments to accommodate different physiologies. For example, the length of the device 10 may be similarly designed to fit substantially inside the cervical canal 12. Variations in length in different embodiments may be used for proper fit of different anatomical characteristics. In one or more embodiments, the device 10 is designed with dimensions to fit much of the intended user population. Based on an average cervical canal length of up to three centimeters and a nominal diameter that spans a few millimeters, the device 10, in this non-limiting example, is designed with a preferred length of three centimeters or less, and five millimeters or less in overall diameter. However, a range of dimensions is possible in different embodiments, e.g., to accommodate different users.

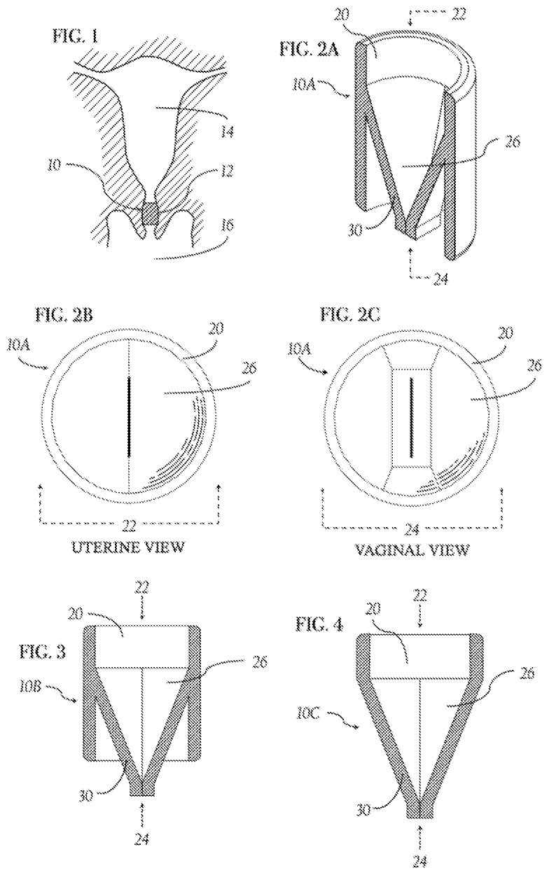

FIG. 2A is a simplified cross-section view illustrating an exemplary intracervical contraceptive device 10A in accordance with one or more embodiments. The device 10A in this example includes a generally tubular, spindle, or cylindrical structure defining a conduit 20 therein extending from an opening at a proximal uterus end 22 of the device to an opening at an opposite distal vaginal end 24 of the device. The structure 20 may be compliant and configured to conform to and securely engage the walls of the cervical canal 12 to inhibit inadvertent movement of the device 10A out of the cervical canal 12. The device 10A may be equipped with a one-way valve 26 shown as a bicuspid valve in the conduit 20, which allows flow of menstrual fluid from the uterine cavity 14 through the conduit 20 to the vagina 16, while inhibiting flow of seminal fluid resulting from coitus from the vagina 16 through the conduit 20 to the uterine cavity 14. In some cases, the one-way valve may be self-closing. Various other types of one-way valves can also be used in the device (e.g., device 10A) in different embodiments including, but not limited to, drain valves, check valves that open and close under spring tension, ball valves that operate within a restricting cage, tricuspid valves that mimic anatomical opening and closing of heart valves, and other valves comprising flaps or as seen in mechanical heart valve implants.

The bicuspid valve 26 may comprise a plurality of flexible flap-like elements 30 oriented to assume a normally closed position. When pressure is applied in one direction, the flap-like elements 30 diverge to create an opening for flow to occur. When pressure is applied in the opposite direction, the flap-like elements 30 are pressed together as shown in the top view (FIG. 2B) and bottom view (FIG. 2C) of the device 10A, to form a liquid-tight seal within its operating parameters. By orienting the valve 26 in the cylindrical structure 20 in the direction permitting flow from the uterus 14 but not intrusion of liquid, including semen, in the reverse direction, contraception is achieved.

An alternative embodiment of device 10 is shown in FIG. 3, wherein device 10B encloses a one-way valve 26 positioned to have the flap-like elements 30 to slightly protrude from the bottom of the cylindrical body 20. Furthermore, another embodiment is shown in FIG. 4, a device 10C includes a one-way valve 26 positioned to have the flap-like elements 30 to completely protrude outside of the cylindrical body 20.

Accordingly, it should be understood that the one-way valve need not be completely contained within the conduit of a device such as described herein. Instead, as these non-limiting examples demonstrate, in some cases, at least portions of the one-way valve need not be within the conduit. In one or more embodiments, a device includes a plurality of one-way valves in tandem within the device conduit.

In one or more further embodiments, a device such as described herein includes spermicidal or hormonal agents therein as further contraceptive safeguards to eliminate sperm viability or fertilization in the event any semen breaches the one-way valve barrier.

As an example, FIG. 5 illustrates an exemplary device 10D in accordance with one or more further embodiments. The device 10D includes a plurality of one-way valves 26 positioned in series and oriented in the same direction inside the cylindrical body 20. Having multiple valves 26 increases the contraceptive reliability by reducing the likelihood of seminal fluid intrusion through the multiple one-way value barriers. In addition, in some cases, one or more spermicidal or hormonal agents may be present, e.g., between the one-way valves.

Devices in accordance with various embodiments may be designed to be located securely in the cervical canal for the intended duration of use wherein the entire device is removable or replaceable as a single unit. For instance, a device may be positioned within a subject for at least a day, at least a week, at least a month, at least 2 months, at least 4 months, at least 6 months, at least 1 year, at least 2 years, at least 3 years, at least 4 years, at least 5 years, etc. The natural compliance of the cervical canal exerts a force on the device 10 to contain the device 10 in normal use. In one or more embodiments, the device 10 is pre-compressed before it is inserted in the cervical canal 12 and allowed to expand once positioned properly to engage with the cervical wall so that it is securely retained in place.

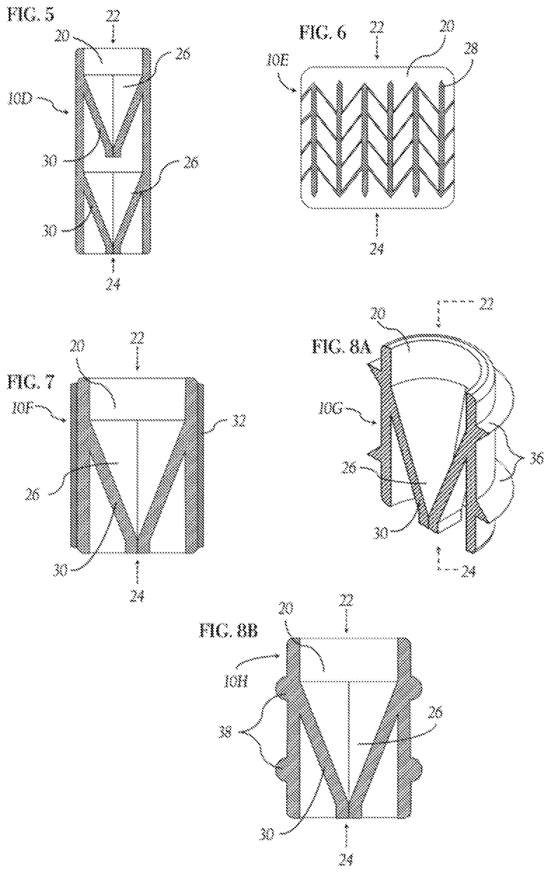

Self-expansion of a device may be achieved by sizing the device in its expanded form according to the size of the cervical canal such that sufficient radial contact force is exerted against the cervical wall to reduce the likelihood of dislocation. In one or more embodiments, as illustrated in FIG. 6, a device 10E includes a self-expanding stent 28 on its outer surface, which adapts to the shape and size of a given user's anatomy once implanted in the cervical canal. When the device 10 needs to be removed or replaced, the stent 28 is contracted using a filament mechanism to compress device 10 thus releasing it from the wall of the cervical canal for retrieval.

In one or more alternate embodiments as shown in FIG. 7, a device 10F is secured within the cervical canal by enhancing adhesion between the outer wall 32 of the device 10F and the tissue lining of the cervix 12. Adhesion may be achieved in some embodiments through nontoxic and biocompatible glues that are applied to the outer surface 32 of the conduit 20 to attach to the cervix 12. In certain embodiments, the material of the device 10F can act as an adhesive in the presence of moisture or pH specific to the cervical tissue. The pH-responsive material may be adaptable in some embodiments to the variance of pH during the menstrual cycle. In one or more embodiments, the device includes one or more features on the outer surface described as quills to help securely anchor a device at a desired location.

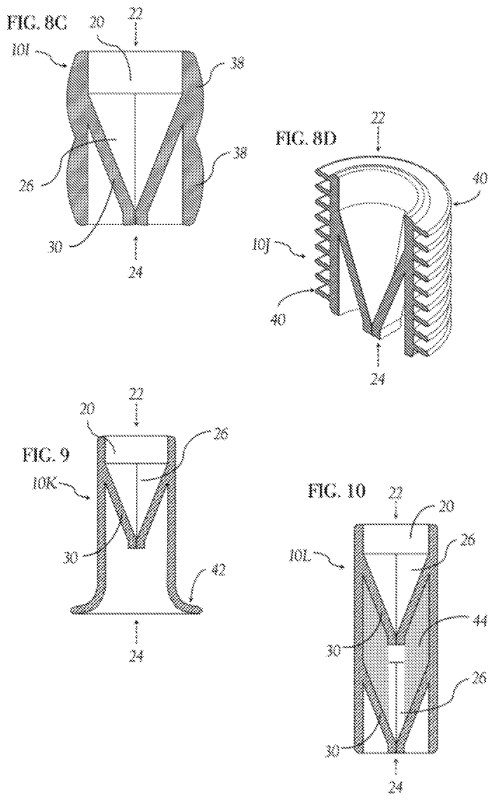

In one or more embodiments, circumferential ridges, bumps, ribs, vanes, etc. can be incorporated on the outside of the cylindrical housing of the device 10 to help secure its position inside the cervical canal. The ribbed texture increases surface may in some cases contact and engage the cervical wall, which may help to anchor a device such as described herein in place. In various embodiments, these external features are configured as unidirectional, bidirectional, or non-directional. In one or more embodiments as shown in FIG. 8, the variations comprising ridges, bumps, ribs, or vanes 36 may wrap around the circumference of the device 10. In one or more embodiments, unidirectional ridges 36 are located but not limited to the proximal or distal ends of the device 10G (FIG. 8A). In some embodiments, the external feature that wraps around the device 10H may have rounded edges 38 (FIG. 8B) and may vary in thickness, location, and length around the device 10I (FIG. 8C). In other embodiments, the external features that wrap about the device 10J represent thin and flexible vanes 40 (FIG. 8D).

As discussed herein, the device 10 can be securely retained inside the cervical canal 12 during its service life in various ways to inhibit upward dislodgement into the uterine cavity 14. As shown in FIG. 9, in one or more alternative embodiments, a device 10K includes a flared or flanged end 42 at the distal vaginal end 24. The flared opening 42 extends out of the opening of the cervix 12 into the vagina 16 with a slight curvature.

A flared opening of a device in accordance with some embodiments may be used to serve one or more purposes, including but not limited to: (1) conforming to the shape of the external os to ensure comfort and minimize exposed edges; (2) limiting the depth of insertion such that the device is properly located inside the cervical canal but does not move into the uterus; and/or (3) preventing migration of the device into the uterus during sexual intercourse due to pressure build up in the vagina.

In some embodiments, including but not limited to those shown in the figures, a pharmacological spermicide is incorporated in the device to enhance contraceptive functionality. Pharmacological spermicide-free embodiments of the device can be left in place in certain embodiments, e.g., with no user intervention or concern over depletion of such agents. In some cases, one-way valve devices enhanced with pharmacological agents such as hormones and spermicides may retain a high degree of physical protection after those agents are depleted, at least according to certain embodiments.

According to certain embodiments, a reservoir 44 to supply impregnated pharmacologic agents may be included in any of the devices 10 disclosed herein as illustrated, e.g., the embodiment of device 10L in FIG. 10. The reservoir 44 may in some cases be used to enhance the contraceptive function of the one-way valve to eliminate sperm viability in the event any semen crosses the one-way valve barrier. Various additives supplied in the reservoir include but are not excluded to spermicides, hormonal agents, and metal-variants.

As shown in FIG. 10, a lining may be provided inside the cylindrical body 20 to function as a hormonal or spermicidal reservoir 44, although other configurations are also possible. In some embodiments (such as the non-limiting example shown in FIG. 102), the lining is in the form of a hollow tube whose lumen permits passage of menstrual discharge.

In one embodiment, the lining is made from a porous matrix that immobilizes the spermicide and allows its release into fluids that pass through the lumen. In another embodiment, the lining is made from a gel-like coating containing the spermicide. By locating this reservoir lining between valves (e.g., between two one-way valves 26, as a non-limiting example), loss of the spermicide may, in some cases, be kept to a minimum. For example, this may occur exclusively in the presence of flow through the device. Furthermore, the spermicide reservoir may be isolated from both upstream and downstream, essentially minimizing contact of the agent with the body, thereby potentially reducing side effects. To the extent the spermicide is located within the cylindrical housing, exposure to the spermicide may still be reduced in some cases even if a single one-way valve is used. However, it should be understood that a spermicide or other agent is not necessarily required.

As non-limiting examples, FIG. 11 shows a one-way valve 26 that may be enhanced by a spermicidal agent or mechanism 44 (for example, where the spermicidal agent is contained within a reservoir), positioned inside the cylindrical body 20 on the vaginal end 24 of the device 10M. FIG. 124 shows a device 10N that includes a one-way valve 26 and a reservoir containing a spermicidal agent 44, positioned in the lining of or otherwise positioned in the cylindrical body 20 on the uterine end 22 of the device 10.

Devices (for example, device 10) that incorporate the spermicidal feature may have predetermined lifetimes in certain embodiments that are governed by their spermicide capacity, although as discussed above, in some embodiments, such devices may continue to function even after the spermicide is exhausted or becomes less or relatively ineffective over time. For example, after extended use and repeated passage of menstrual fluids, the level of spermicide may gradually diminish until it is insufficient to inactivate errant sperms. At that point, the device may be replaced with a new unit (or at least a portion of the device replaced, which may be a portion of the device containing spermicide; for example, the device may contain a first part and a second part as was discussed herein), or continue to be used without the spermicidal feature, e.g., while retaining the ability to block physical entry of sperm.

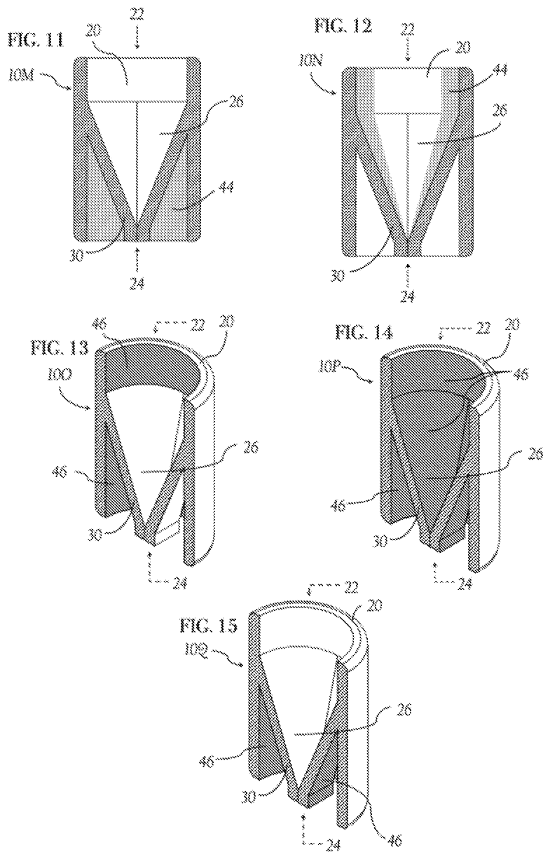

In other embodiments, copper is incorporated in the device, for example, for a more durable spermicidal effect. The copper may be included in a reservoir, e.g., as shown in the non-limiting examples of FIG. 10-12. In yet another aspect, a device with virtually permanent spermicidal capability is constructed by combining the unidirectional flow valve and a spermicidal lining containing elemental copper. Copper has durable spermicidal properties as well as preferable anti-microbial properties to reduce or eliminate infection.

In some embodiments, the inner surface of a device such as described herein may be lined with a matrix made from copper, or a carrier matrix in which copper is dispersed. In this way, copper may be exposed to the fluid that passes through the device. Menstrual fluid may in some cases pass through the device unaffected (or since the menstrual fluid is to be expelled, regardless, any changes may not be particularly problematic to the subject). However, seminal fluid that flows over the copper surface may be affected by the copper, for example, losing some or all of its fertilizing properties. A significant advantage over certain prior art, especially IUD devices that incorporate copper, is the absence of direct contact between cervical or uterine tissue and the copper component. The result may be a reduction of adverse reactions associated with the use of copper IUDs such as inflammation, cramps, or heavier periods.

The copper may be present, in some embodiments, as a copper-lined hollow cylinder situated in the cervical canal. For example, in one embodiment as shown in FIG. 13, as yet another non-limiting example, the durable spermicidal feature of a copper-lined hollow cylinder 46 situated in the cervical canal 12 constitutes a self-sufficient contraceptive device 100 enclosing the one-way valve 26.

In another embodiment, the spermicidal feature of a copper-lined conduit 46 complements a copper-lined unidirectional valve 26 illustrated in FIG. 14 to yield a device 10P to be situated in the cervical canal 12. As this non-limiting example shows, some embodiments are generally directed to devices comprising a copper-lined conduit and/or a copper-lined unidirectional valve. Devices such as these may prevent or minimize semen intrusion into the uterus while allowing passage of menstrual fluid and can offer an additional safeguard to inactivate sperms that may breach the unidirectional valve. The result shown in this particular example is a contraceptive system of enhanced efficacy, for example, with no expiration period in certain cases.

In another non-limiting example embodiment in FIG. 15, the copper-lining 46 is exclusive to the distal end 24 of the device 10Q. Thus, in some embodiments, the device may be constructed to minimize the interaction of the copper component and the cervical and uterine tissues, to preserve the health of the tissue, and/or to localize the spermicidal properties of the copper.

In variations of the presented embodiments, the copper component may comprise a metallic mesh of copper. In some embodiments, the copper component comprises a composite of dissimilar fibrous mesh, one of which is copper. In some embodiments, the copper component comprises a coating of copper particles, preferably micronized, on a carrier matrix of polymeric fibers or network. In some embodiments, the copper component is a sintered structure. Other techniques for including copper within the device may also be used in still other embodiments.

Contraceptive devices in accordance with various embodiments may be biocompatible in certain embodiments, e.g., to avoid foreign body response and/or ensure user safety and comfort. Physiological compatibility between the device and cervical canal is attained in accordance with some embodiments by fabricating at least a portion of the device by using known biocompatible materials. In another set of embodiments, compatibility may be attained by constructing at least a portion of the device using a composite material with suitable mechanical properties for housing the valve. In some cases, at least a portion of the device may be coated or treated with a biocompatible material, for example, one which does not itself have the requisite mechanical properties of a durable, stable housing.

Various materials can be used to construct devices such as any of those described herein, according to some embodiments. In one or more embodiments, at least a portion of the device may be constructed from various polymers that satisfy conditions such as suitable compliance, strength, durability, and biocompatibility criteria. In one or more embodiments, at least a portion of the device can be constructed from materials generally recognized as safe from commercialized or predicate devices. In one or more embodiments, the material properties of a device may be hydrophobic, e.g., to prevent clumping and adhesion of menstrual flow to the device wall. In some embodiments, the materials may be non-chemotactic, e.g., to avoid chemical stimulation that can result in an inflammatory response. In some embodiments, the materials may be non-traumatic, e.g., to preserve tissue health of the cervix and surrounding anatomy. In some embodiments, the materials may be compliant and crack-resistant, e.g., to withstand the regular contractions of the cervix. In some embodiments, the materials may be flexible and conformal, e.g., to allow for form-fitting to variations in anatomy size and shape. In some embodiments, the materials may be thermo-responsive, e.g., such that they change configuration or shape as a function of body temperature. In some embodiments, the materials may be dielectric, e.g., to allow transmission of electric force without conduction. In some embodiments. the materials may be radiopaque such that their position in the body can be monitored non-invasively by appropriate instruments.

In various embodiments, the material of the conduit may differ from the material of the valve. In one or more embodiments, at least a portion of a device such as described herein may be made of polycarbonate-urethane which may, for example, impart one or more of flexibility, durability, compliance, and high crack resistance of a device. In one or more embodiments, as another example, at least a portion of the device may be made of a silicone or a silicone-based elastomer, for example to impart one or more of the stated criteria. In one or more embodiments, at least a portion of a device may be made of a hydrogel, for example, for example, to impart one or more the stated criteria. In one or more embodiments, at least a portion of the device may be made of one or more metallic materials, including but not limited to cobalt, chrome, gold, or silver, e.g., which may be able to enhance the contraceptive ability of the device, at least in certain embodiments. In one or more embodiments, at least a portion of a device may incorporate biodegradable, and/or resorbable materials. In one or more embodiments, at least a portion of the device may incorporate examples of thermoplastic materials that are widely deployed in medical devices. In one or more embodiments, at least a portion of the device may incorporate exclusively or a combination of inorganic or organic elastomers, or inorganic, organic, ionic, amphoteric, liquid crystal, or synthetic macromolecules. In one or more embodiments, at least a portion of the device may be constructed from elastomers exemplified by polydimethylsiloxane, polyurethane, their copolymers, blends, or compounds with a large variety of polymers to impart the necessary compliance, strength, durability, and various biocompatibility criteria.

To achieve the conformal properties, materials of construction may be selected in certain embodiments with certain degrees of flexibility and compliance. Softer materials are generally preferred to enhance comfort for the wearer. However, in some embodiments, the valve may be protected from distortion by compressive forces exerted on the device housing. In one embodiment, one-way valves are selected that feature a rigid body from a first material, and flexible flaps with a second elastomeric material. In an alternative embodiment, valves constructed from a single, relatively soft material (body and flaps) are reinforced with an insert made from a second rigid material. The insert, in the form of a sleeve or ring, may be concentrically located in close proximity to the valve flaps to prevent distortion of the valve and its flaps under radial compression when the device is in use. In this way, the valve movement is unimpeded even in devices made of relatively soft materials to ensure comfort even for extended wear.

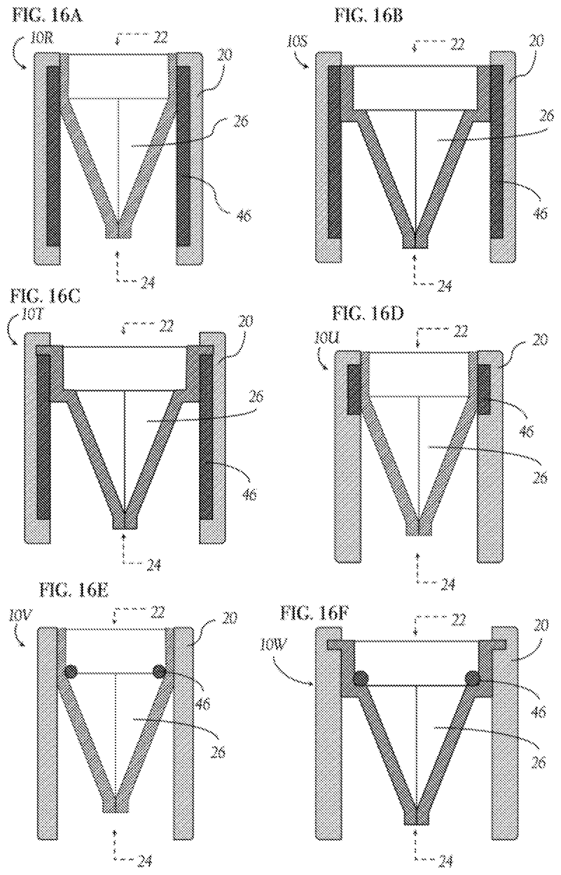

In one non-limiting embodiment, as shown in FIG. 16A, the insert spans the length of the cylindrical body 20. The elongated insert provides coverage along the entire length of the valve and can be incorporated into the cylindrical body 20 using different forms of valves 26, as shown in FIG. 16B and FIG. 176C. The insert can also be shortened to protect the intersection of the valve 26 and the cylindrical body 26, as shown in FIG. 16D. Through yet another non-limiting embodiment, the insert can be representative of a ring 46 that protects the valve 26 from radially compression, as illustrated in FIG. 16E and FIG. 16F using various valve-designs.

Suitable materials for the reinforcing insert comprise metals such as stainless steel, titanium, copper, silver, non-metals such as ceramics, biocompatible engineering polymers, or their composites. Differing surface and material properties of materials used in different embodiments can be used in accordance to achieve optimized mechanical performance and biocompatibility, in accordance with a variety of embodiments as discussed herein. In one or more embodiments, the materials utilize surface structures that can be smooth, textured, microporous, and/or nanoporous, for example, to control the interaction between physiological material and the surface, e.g., to manage risk of infection and/or foreign body response.

Devices of certain aspects of the present disclosure may be deployed in operations similar to current practice associated with deploying IUDs, or other medical devices that are inserted using transcatheter implantation procedural methods. In one or more embodiments, insertion of the device is performed by healthcare providers using common gynecological equipment and procedures. Insertion aids are used in certain embodiments to facilitate deployment of the device. Healthcare providers already familiar with placing and maintaining IUDs are well prepared to service users of the present devices. Below are some equipment and procedural considerations for deployment. However, it should be understood that these are presented by way of example only, and that in other embodiments, other methods may be used for deployment.

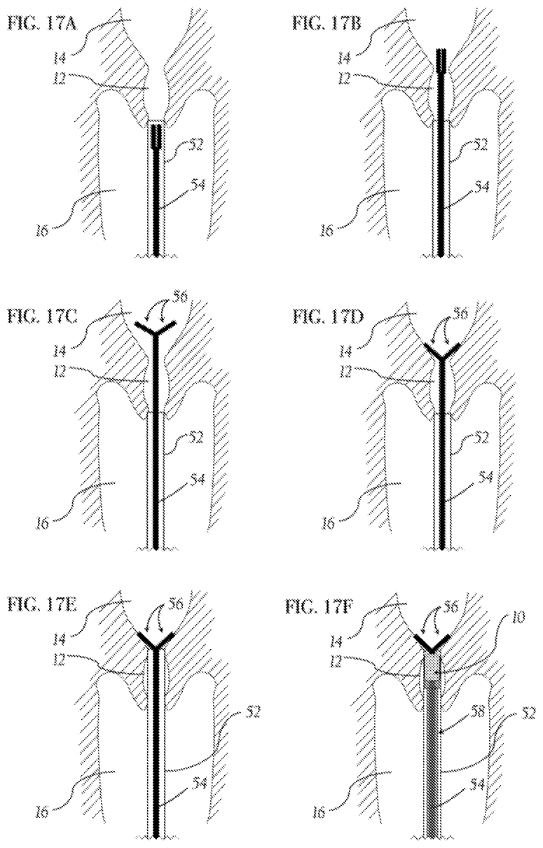

In one embodiment, the device may be inserted into the cervical canal, without necessarily spanning or intruding the uterine cavity. In one non-limiting example of a method of use, the device 10 is deployed with a delivery system similarly to a stent, as shown in FIG. 17. The device is compressed within an outer sheath of a delivery device and expanded to the size of the cervical canal after deployment in a manner similar to the deployment of stents in blood vessels. The outer sheath 52 enters the vaginal canal 16 to the base of the cervix 12 (FIG. 17A). A guidewire 54 is fed through the outer sheath 52 and is inserted through the cervical canal 12 (FIG. 17B). The guidewire 54 is extended upward into the uterine cavity 14 where two prongs 56 are exposed (FIG. 17C). The guidewire 54 is gently pulled back towards the vagina 16 until the prongs interact with the base of the uterus 14 and act as a measurement tool to indicate the top of the cervix 12 (FIG. 17D). The outer sheath 52 is fed up along the guidewire (FIG. 17E). The device 10 is then fed within the outer sheath 52 along the guidewire 54 using a pushing piston 58 (FIG. 17F). The outer sheath 52 and the guidewire 54 are then pulled back to expose the device 10 that is held in position with the pushing piston 58 (FIG. 17G). Once the device 10 is self-expanded the pushing piston 58 is withdrawn with the rest of the delivery system (FIG. 17H).

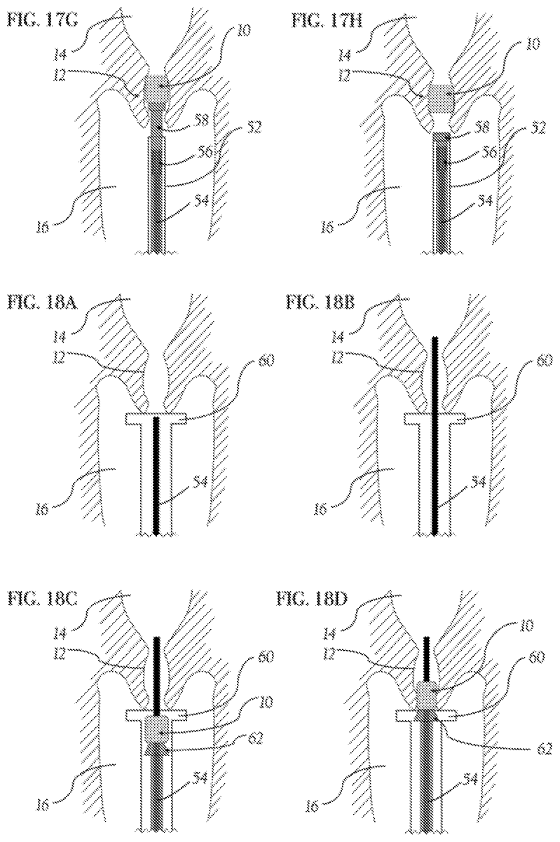

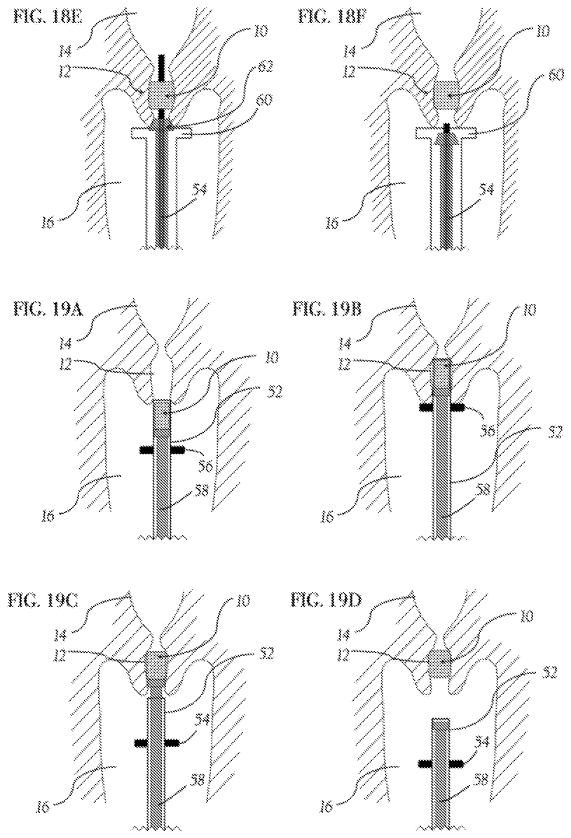

In another example of a method of use, the device 10 is deployed with a delivery system, as depicted in FIG. 18. The cylindrical insertion tube 60 acts similarly to the barrel of a syringe and is designed to fit over the rounded extremity of the cervix 12. In this way. the center of the insertion tube is aligned with the ostium or external os (FIG. 18A). A guidewire 54 is fed through the insertion tube through the cervix 12 and into the uterine cavity 14 (FIG. 18B). The device 10 is then fed through the insertion tube 60 along the guidewire 54 (FIG. 18C), and into the cervical canal 12 by means of a plunger 62 (FIG. 18D). The plunger 62 pushes the device 10 past the junction between the cervix 12 and the vagina 16, and conformally expands within the cervical canal (FIG. 18E). One the device 10 is secured, the delivery system can be removed (FIG. 18F). In one embodiment of this delivery method, alignment of the delivery system is reliant on the position of the insertion tube 60 at the extremity of the cervix 12, e.g., exclusively reliant in some cases, such that the device 10 is designed to be properly located when the flared end of the device is seated on the ostium. As with IUD deployment, a speculum may be used to increase access and assist in preparing the cervical area for this procedure. The insertion tube 60 may be designed for initial placement and subsequent re-insertion by a qualified healthcare provider.

In another non-limiting example of a method of use, the device 10 is deployed in a similar manner to FIG. 17 but without the guidance of a guidewire, as shown in FIG. 19. This delivery system does not extend through the internal os nor into the uterine cavity. The outer sheath 52 enters the vaginal canal 16 to the base of the cervix 12 (FIG. 19A). In some embodiments, the delivery system includes a stopper 56 that is intended to prevent the delivery system from passing beyond a predetermined length from the ectocervix and into the cervical canal 12 (FIG. 19B), preventing passage through the internal os and into the uterine cavity 14. The outer sheath 52 is then pulled back to expose the device 10 that is held in position with the pushing piston 58 (FIG. 19C). Once the device 10 is inserted the pushing piston 58 is withdrawn with the rest of the delivery system (FIG. 19D). In another embodiment, the pushing piston 58 is retracted from the reproductive system prior to the outer sheath 52 as well as in yet another distinction, the pushing piston 58 and the outer sheath 52 are retracted at the same time.

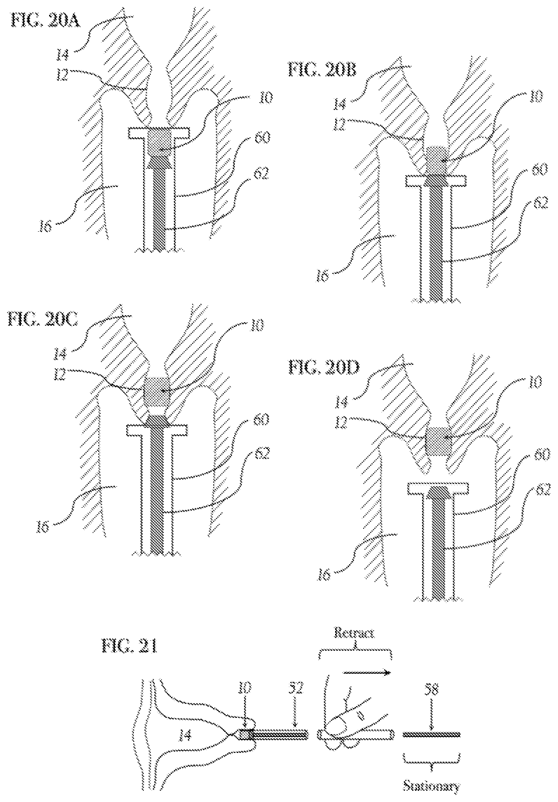

In yet another example of a method of use, the device 10 is deployed in a similar manner to FIG. 18 but without the guidance of a guidewire, as depicted in FIG. 20. The cylindrical insertion tube 60 acts similarly to the barrel of a syringe and is aligned with the ostium or external os (FIG. 20A). The plunger 62 pushes the device 10 past the junction between the cervix 12 and the vagina 16, and conformally expands within the cervical canal (FIG. 20B). The plunger 62 is then retracted leaving the device expanded in place (FIG. 20C). Once the device 10 is secured, the delivery system can be removed (FIG. 20D). FIG. 21 illustrates an additional perspective of the insertion approach of the device into the cervix, which is representative of FIG. 19.

In some embodiments, a device such as those described herein may be maintained using simple procedures, such as using sheathed tampons, by constructing the insertion tube with a semi-rigid polymer with a rounded end and slitted such that the normally closed round shape of the insertion tube opens to allow the device to be pushed out by the plunger. This aspect may be helpful at improving adoption among subjects, e.g., since there may be initial oversight, but subsequent independence, from medical professionals in some cases. In other use cases, devices such as those described herein can be deployed and serviced entirely through healthcare providers, e.g., with little or no user involvement.

In an aspect of the present disclosure, a string may be built into the body of a device in accordance with certain embodiments such that it extends into the vagina after the device is fully inserted into the cervical canal. In this way, a device may be removed by pulling on the string. In one embodiment, the string can be molded into a cylindrical housing of a device. In another embodiment, the string may comprise a monofilament that does not retain liquid by capillary action. This may be useful, for example, to reduce the risk of bacteria growth and adverse consequences therefrom. In another embodiment, the string comprises a shape memory polymer. The part of the string that extends into the vagina, in some embodiments, may be pre-formed into a coil designed to loosely surround the opening of the cervix. In some cases, the string does not protrude into the vagina. In certain cases, the string is able to self-retract into a coil, e.g., after a device is inserted. In some embodiments, the string stays in place due to the shape memory of the material. In some embodiments, the string is accessible if the device needs to be removed by a trained health professional or by self-removal, and optionally cannot be felt during sexual intercourse and/or be easily reached by the wearer. In some embodiments, devices such as those described herein may be retrieved using current techniques of removing IUDs or offer certain advantages over current retrieval methods. Below are a few non-limiting procedural considerations.

In one or more embodiments, the retrieval of a device such as described herein can be aided by a retrieval string or filament. This may be attached as described previously, or on the distal end of a device, etc. After inserting a device, in some cases, the retrieval filament may be trimmed to a suitable length to fit inside the vagina. The removal of a device through the use of retrieval filaments may in some cases be accomplished using common gynecological procedures and/or be aided by forceps or other tools. In some cases, the retrieval may be performed by a healthcare provider. The provider or the user, in some cases, can monitor the presence of a device by accessing the retrieval filament as needed. In some cases, this can be achieved without pulling on the filament to avoid dislocation.

In one or more embodiments, a device such as described herein may be removed by inserting forceps to directly grasp the edge of the device at the opening of the cervix. Collapsing a device, in accordance with some embodiments, can help dislodge the device from the cervical and facilitate its retrieval. In addition, in some cases, collapsing a device may break the pressured seal and subsequently allow for case of retrieval.

In some embodiments, retrieval or retrieval of a device can be cased using a release agent to reduce the adhesion between the device and the tissue of the cervical canal and can be administered by the user on demand. The release agent may access the cervix by the vaginal canal and may dissolve an adhesive used to attach the device to the cervical wall, for example, in embodiments where an adhesive is used.

In some embodiments wherein the materials of a device are biodegradable, the removal of the device may be through degradation, e.g., with a biocompatible agent that may be administered by the user or another person, e.g., a healthcare provider. The removal agent may be delivered to the vaginal canal, e.g., in a similar manner to inserting a tampon or other topical gel in pharmaceutical applications.

Devices in accordance with various embodiments overcome disadvantages of certain prior art contraceptive devices and techniques, including other LARCs and surgical interventions. In some cases, the devices in accordance with various embodiments are safe and reliable. They can be used, e.g., on a temporary or permanent basis, and in some cases are removable to enable pregnancy.

Devices in accordance with various embodiments can be inserted in the cervical canal by a medical professional in a minimally invasive procedure, with minimum discomfort to the user and typically no requirement of a follow-up or procedure to reaffirm placement or retrieve the device. Additionally, devices in accordance with various embodiments can be advantageously used in developing countries, which have generally prioritized sterilization procedures to avoid socio-economic adversities associated with unintended pregnancies, by providing a long-term birth control option that allows for natural physiological menstrual bleeding, is insertable virtually anywhere, and is reversible to accommodate users who desire or would like the option to conceive in the future.

One advantage of some devices in accordance with various embodiments over prior art devices is that it avoids complexities of planning and preventing seminal fluid inflow that accompany short-term contraception (e.g., birth control pill, patch, and ring) and single-use devices (e.g., condoms). The one-way mechanical valve with or without pharmacological or copper spermicidal features avoids complexities that accompany hormone-dependent devices (e.g., IUDs and transdermal implant), surgical operations, which are typically high risk and high-cost procedures, inflammatory responses (as seen, e.g., with the copper IUD), and fibrous scar tissue formation associated with surgical fallopian tubal occlusion.

Another advantage of certain devices in accordance with various embodiments is that their use involves a single procedure, which avoids complications of multiple procedures and follow-up visits typical of other birth control devices. The insertion method may initially be performed by a healthcare professional, optionally followed by elective training to enable the user to manage device retrieval and replacement on her own. Once properly trained, the user would gain wide latitude to manage her fertility in a safe manner virtually anywhere, not restricted to a clinician's office or hospital operating room setting. This approach is inherently scalable because it embodies the advantages of LARC while overcoming much of its remaining drawbacks, resulting in the safest, most convenient contraception technology demanded by a global solution.

U.S. Provisional Patent Application Ser. No. 63/296,192, filed Jan. 4, 2022, entitled “Conformal Contraception Devices and Methods,” is incorporated by reference herein in its entirety.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control. All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of.” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of.” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An intracervical conformal contraceptive device, comprising:
a housing having a cervical shape, defining a conduit passing from a first proximal end to a second distal end of the housing, wherein the housing comprises a one-way valve within the conduit, the one-way valve allowing fluid to pass from the first proximal end to the second distal end.

2. The device of claim 1, wherein the housing is substantially tubular.

3. The device of claim 1 wherein the housing is substantially spindle-shaped.

4. The device of claim 1, wherein the device has a length of no more than 3 cm.

5. The device of claim 1, wherein the device has a diameter of no more than 12 mm.

6-7. (canceled)

8. The device of claim 1, wherein the material housing comprises a polymer.

9. (canceled)

10. The device of claim 1, wherein the housing comprises a polycarbonate.

11. The device of claim 1, wherein the housing comprises a polyurethane.

12. The device of claim 1, wherein the housing comprises a silicone.

13. The device of claim 1, wherein the housing comprises a hydrogel.

14-18. (canceled)

19. The device of claim 1, further comprising a spermicidal agent positioned in the housing.

20-23. (canceled)

24. The device of claim 1, wherein the housing comprises a flange at the first end of the device.

25. The device of claim 1, wherein the housing comprises a flange at the second end of the device.

26. A method, comprising:
inserting, into a cervix of a subject, a device comprising a housing defining a conduit passing from a first end to a second end of the housing, the housing comprising a one-way valve within the conduit, such that the one-way valve is positioned to allow fluid to exit the vagina.

27-47. (canceled)

48. The method of claim 26, wherein the housing comprises a retention mechanism.

49. The method of claim 48, wherein the retention mechanism comprises one or more circumferential vanes.

50. The method of claim 48, wherein the retention mechanism comprises one or more ridges.

51. (canceled)

52. The method of claim 26, wherein the housing comprises a textured surface.

53-90. (canceled)
